(19) United States
(12) Patent Application Publication
(10) Pub. No.: US 2015/0065915 A1
(43) Pub. Date: Mar. 5, 2015

(54) EndoBrush with Receptacle

(71) Applicant: Farrukh M. Jafri, Fredericksburg, VA (US)

(72) Inventor: Farrukh M. Jafri, Fredericksburg, VA (US)

(21) Appl. No.: 14/469,383

(22) Filed: Aug. 26, 2014

Related U.S. Application Data
(60) Provisional application No. 61/870,702, filed on Aug. 27, 2013.

(51) Int. Cl. A61B 10/04; A61B 1/273

(52) U.S. Cl. CPC A61B 10/04; A61B 1/2736; A61B 2010/0216
USPC 600/569

(57) ABSTRACT

A tissue-sampling brush is a medical device for acquiring internal patient tissue samples utilizing conventional endoscopic devices. The tissue-sampling brush features an elongated body that may be inserted into the channel of an endoscopic device and manually manipulated by a handle. The tissue-sampling brush is sleeved in a sheath during insertion into the patient's body with the plurality of bristles deploying from the sheath during sample acquisition. The sheath facilitates insertion and removal of the tissue-sampling brush into the endoscopic device as well as structural support and protection for the plurality of bristles and endoscopic device. A plurality of bristles is scraped against the interior walls of the patient's body in order to acquire a tissue sample. After acquisition, the plurality of bristles is retracted back into the sheath and any trickling sample is captured by a receptacle located adjacent to the plurality of bristles.

The current application claims a priority to the U.S. Provisional Patent application Ser. No. 61/870,702 filed on Aug. 27, 2013.

### FIELD OF THE INVENTION

The present invention relates generally to a medical device for obtaining a patient tissue sample. More specifically, the present invention is a tissue-sampling brush with receptacle for improved acquisition and retention capabilities of patient tissue samples during a medical procedure such as a gastrointestinal endoscopy involving internal sample acquisition.

### BACKGROUND OF THE INVENTION

The gastrointestinal endoscopy is a medical procedure during which a medical professional is able to view the interior of a patient's upper and lower gastrointestinal tracts. A medical device known as an endoscope (imaging system) is generally inserted into the patient's body in order to search for abnormalities and findings that are indicative of medical conditions such as ulcers, cancerous/precancerous growths, inflammation, and infection. These medical conditions may result in a variety of symptoms including abdominal pain, nausea, vomiting, and difficulty in swallowing. During the course of a gastrointestinal endoscopy, it is common for the medical professional to acquire patient tissue and mucus samples for testing. This is typically done by inserting a sample-acquisition device into the patient's gastrointestinal tract. The sample-acquisition device often contains a brush with bristles. The insertion of the sample-acquisition device is aided by housing the device within a sheath that additionally serves to provide structural support for the device. The sheath is inserted into a corresponding channel within an endoscope in a manner such that the brush bristles are able to protrude from the sheath. The sample-acquisition device is manually manipulated in order to allow the bristles to scrape against the interior walls of the gastrointestinal tract, thus obtaining a sample. After the sample has been acquired, the brush is retracted back into the sheath and the endoscope is removed from the patient's body. A common problem that arises during sample acquisition is the tendency for a large portion of the sample to become lost due to the fact that the bristles of the brush are of a larger diameter than that of the sheath. This often causes a large portion of the acquired sample to trickle towards the far end of the brush and become lost during retraction of the brush back into the sheath. The present invention seeks to address the aforementioned issues as well as provide an improved means of acquiring a tissue sample during the course of an endoscopic procedure.

The present invention is a tissue-sampling brush with improved sample acquisition and retention capabilities. The present invention is primarily intended for use during patient tissue sample acquisition during a medical procedure such as a gastrointestinal endoscopy. However, the present invention has applications in virtually any medical field and procedure involving endoscopy and/or internal tissue sample acquisition. The present invention seeks to improve upon patient tissue sample retention through the implementation of a receptacle that is designed to collect and capture most, if not all, of an acquired patient tissue sample.

### BRIEF DESCRIPTION OF THE DRAWINGS

FIG. 1 is a perspective view of the elongated body, receptacle, and handle of the present invention.

FIG. 2 is a perspective view of the sheath of the present invention.

FIG. 3 is a front view of the present invention in a deployed configuration.

FIG. 4A is a front internal view of the present invention in a deployed configuration.

FIG. 4B is a detail view of the receptacle and brush portion of the present invention.

FIG. 5 is a front view of the present invention in a retracted configuration.

FIG. 6 is a front internal view of the present invention in a retracted configuration.

### DETAIL DESCRIPTIONS OF THE INVENTION

All illustrations of the drawings are for the purpose of describing selected versions of the present invention and are not intended to limit the scope of the present invention.

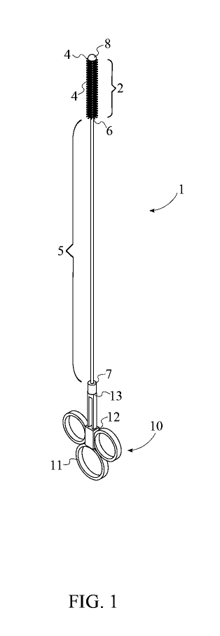

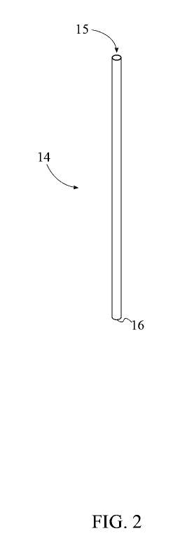

The present invention is a tissue-sampling brush for use in acquiring a patient sample during gastrointestinal endoscopies and similar medical procedures. The present invention comprises an elongated body 1, a receptacle 8, a handle 10, and a sheath 14. The elongated body 1, the receptacle 8, and the handle 10 are shown in FIG. 1 while the sheath 14 is shown in FIG. 2. The receptacle 8 is positioned opposite to the handle 10 across the elongated body 1. The sheath 14 sleeves various portions of the elongated body 1 during use of the present invention. The elongated body 1 allows the present invention to be utilized in conjunction with conventional endoscopic devices. The receptacle 8 provides improved sample acquisition and retention capabilities while the handle 10 allows manual manipulation of the present invention during use. Finally, the sheath 14 facilitates entry and exit of the present invention into and out of the patient's body and additionally provides structural support for the present invention. Because the present invention is primarily intended for internal medical applications, in the preferred embodiment of the present invention, the elongated body 1, the receptacle 8, the handle 10, and the sheath 14 are composed of a hypoallergenic material.

The elongated body 1 comprises a brush portion 2 and a neck portion 5. The neck portion 5 comprises a first end 6 and a second end 7. The brush portion 2 is connected adjacent to the first end 6. The receptacle 8 comprises a concave opening 9. The receptacle 8 serves as a temporary container for holding an acquired patient sample following the sample acquisition process utilizing the brush portion 2. As such, the receptacle 8 is positioned adjacent to the brush portion 2 opposite to the first end 6. The concave opening 9 is oriented towards the brush portion 2. This aids the concave opening 9 in retaining all or the majority of the acquired sample. The receptacle 8 is preferably composed of a material that is sufficiently rigid to prevent the receptacle 8 from inverting during retraction back into the sheath 14.

The brush portion 2 comprises an elongated base 3 and a plurality of bristles 4. The plurality of bristles 4 is radially positioned about the elongated base 3. Each of the plurality of bristles 4 is positioned in close proximity to the remaining bristles in order to aid in acquiring a tissue sample from the within the patient's body. The plurality of bristles 4 is preferably composed of a soft material in order to prevent damage to the interior walls of the patient's body. However, the plurality of bristles 4 is preferably sufficiently rigid as well in order to maintain structural integrity of the bristles during use of the present invention. The plurality of bristles 4 is radially positioned about the elongated base 3 and is additionally evenly distributed along the elongated base 3. The radial and even distribution of the plurality of bristles 4 allow for multiple means of acquiring a patient sample. The radial positioning of the plurality of bristles 4 about the elongated base 3 allows for the present invention to be rotated in order to acquire a patient sample. The even distribution of the plurality of bristles 4 along the elongated base 3 allows the present invention to be actuated back and forth in order to acquire a patient sample.

The handle 10 is connected adjacent to the second end 7 and is utilized to manually manipulate the present invention during use. The handle 10 comprises at least one finger loop 11, a loop hub 12, and a stem 13. The stem 13 is connected adjacent to the second end 7 and serves as a connector between the second end 7 and the loop hub 12. As such, the loop hub 12 is connected adjacent to the stem 13 opposite to the second end 7. The at least one finger loop 11 is connected about the loop hub 12. The number of finger loops of the handle 10 may vary across multiple embodiments of the present invention. The user may insert a finger through the at least one finger loop 11 in order to maintain control of the present invention during use. Once the present invention has been inserted into the patient's body and maneuvered to the desired sample-acquisition site, the brush portion 2 is advanced into contact with the interior walls of the patient's body. The present invention is then manually manipulated via the handle 10 in order to allow the plurality of bristles 4 to scrape the interior walls of the patient's body to acquire a sample.

The sheath 14 comprises a first open end 15 and a second open end 16 with the first open end 15 and the second open end 16 being positioned opposite to each other along the sheath 14. The elongated body 1 is sleeved by the sheath 14 during use of the present invention.

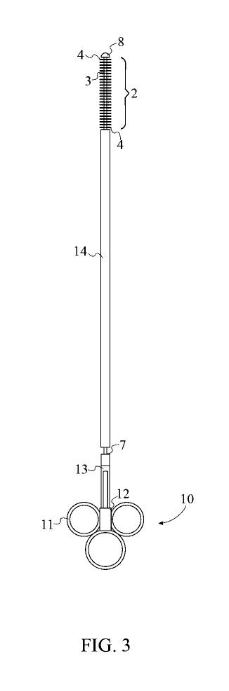

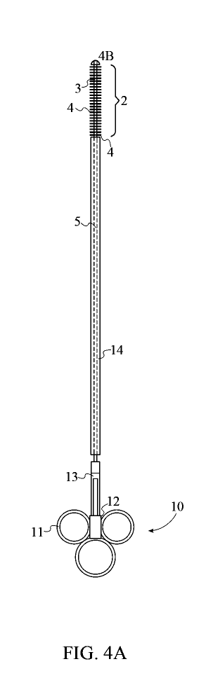

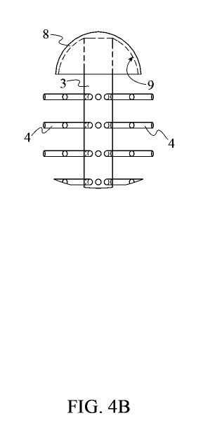

The present invention is utilized in two primary configurations including a deployed configuration and a retracted configuration. The deployed configuration, is shown in FIG. 3 and FIG. 4A. A magnified view of the receptacle 8 and the brush portion 2 is shown in FIG. 4B. In the deployed configuration, the brush portion 2, the neck portion 5, the handle 10, and the sheath 14 are positioned such that the brush portion 2 and the receptacle 8 are advanced from the sheath 14. The brush portion 2 is externally positioned to the sheath 14 and is positioned adjacent to the first open end 15. The first open end 15 is positioned opposite of the receptacle 8 along the brush portion 2. Additionally, the second open end 16 is positioned adjacent to the handle 10.

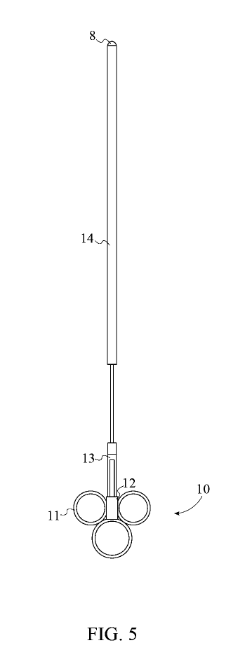

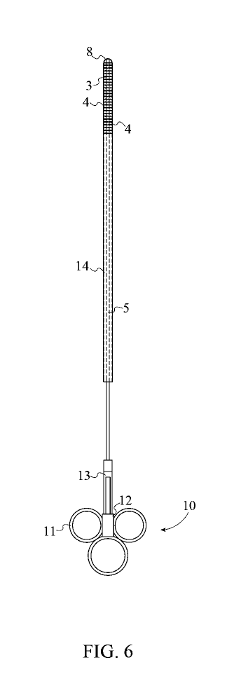

In the retracted configuration, only the receptacle 8 remains externally positioned to the sheath 14. The retracted configuration is shown in FIG. 5 and FIG. 6. In the retracted configuration, the brush portion 2, the neck portion 5, the handle 10, and the sheath 14 are positioned such that only the receptacle 8 is extended from the sheath 14. The brush portion 2 is internally positioned to the sheath 14. The receptacle 8 is positioned adjacent to the first open end 15 while the second open end 16 is positioned adjacent to the handle 10.

The object of the present invention is to facilitate sample acquisition and retention with relation to an internally acquired patient tissue sample. The present invention is designed for use in conjunction with conventional endoscopic devices. During sample acquisition, the present invention is inserted into the accessory channel of an endoscopic device that is inserted into the patient's body. During insertion of the present invention, the present invention remains in the retracted configuration with only the receptacle 8 remaining externally positioned to the sheath 14. In the retracted configuration, the sheath 14 is able to cover the brush portion 2 in order to prevent damage to the plurality of bristles 4 and/or the endoscopic device channel. Once the endoscopic device and the present invention have reached the sample site within the patient's body, the brush portion 2 is advanced from the first open end 15. The user may grasp the at least one finger loop 11 to maneuver the present invention. The extended brush portion 2 is placed into contact with the interior walls of the patient's body. The user may then manually manipulate the present invention in two ways to acquire a tissue sample. The present invention may be rotated in order to allow the radially positioned plurality of bristles 4 to scrape against the interior walls of the patient's body. The plurality of bristles 4 may be scraped against the interior walls of the patient's body by actuating the present invention in a back and forth motion. Once a sample has been acquired, the brush portion 2 is retracted back into the sheath 14. During traditional endoscopic tissue sample acquisition, the retraction process causes the captured tissue sample and mucus to trickle down and become lost. This is primarily due to the fact that the diameter of the brush portion 2 is often larger than that of the sheath 14. The increased diameter of the brush portion 2 is necessary for adequate scraping of the interior walls of the patient's body, but the increased diameter is not conducive for retaining an acquired tissue sample. During retraction of the brush portion 2 back into the sheath 14, the acquired sample tends to dislodge from the plurality of bristles 4. However, use of the present invention in lieu of traditional endoscopic sample acquisition devices allows for the retention of most, if not all, of an acquired sample following the retraction process. The receptacle 8 is able to capture any trickling tissue sample for subsequent transfer to a sample slide prior to sample analysis. Alternatively, the brush portion 2 may be cut and removed from the tissue-sampling brush altogether prior to sample analysis.

The present invention is primarily intended for use in gastrointestinal endoscopic sample acquisition. However, the present invention has additional applications in virtually any medical field and procedure involving endoscopy and/or internal sample acquisition. As such, the present invention may be utilized by a wide variety of medical professionals including, but not limited to, pulmonologists and gynecologists.

Although the invention has been explained in relation to its preferred embodiment, it is to be understood that many other possible modifications and variations can be made without departing from the spirit and scope of the invention as hereinafter claimed.

What is claimed is:

1. A tissue-sampling brush comprises:
an elongated body;
a receptacle;
the elongated body comprises a brush portion and a neck portion;
the neck portion comprises a first end and a second end;
the brush portion being connected adjacent to the first end;
the receptacle comprises a concave opening;
the receptacle being positioned adjacent to the brush portion opposite to the first end; and
the concave opening being oriented towards the brush portion.

2. The tissue-sampling brush as claimed in claim 1 further comprises:
the brush portion comprises an elongated base and a plurality of bristles;
the plurality of bristles being radially positioned about the elongated base; and
the plurality of bristles being evenly distributed along the elongated base.

3. The tissue-sampling brush as claimed in claim 1 further comprises:
a handle; and
the handle being connected adjacent to the second end.

4. The tissue-sampling brush as claimed in claim 3 further comprises:
the handle comprises at least one finger loop, a loop hub, and a stem;
the stem being connected adjacent to the second end;
the loop hub being connected adjacent to the stem opposite to the second end; and
the at least one finger loop being connected about the loop hub.

5. The tissue-sampling brush as claimed in claim 1 further comprises:
a sheath;
the sheath comprises a first open end and a second open end;
the first open end and the second open end being positioned opposite to each other along the sheath; and
the elongated body being sleeved by the sheath.

6. The tissue-sampling brush as claimed in claim 5 further comprises:
wherein the brush portion, the neck portion, a handle, and the sheath are in a deployed configuration;
the brush portion being externally positioned to the sheath;
the brush portion being positioned adjacent to the first open end;
the first open end being positioned opposite of the receptacle along the brush portion; and
the second open end being positioned adjacent to the handle.

7. The tissue-sampling brush as claimed in claim 5 further comprises:
wherein the brush portion, the neck portion, a handle, and the sheath are in a retracted configuration;
the brush portion being internally positioned to the sheath;
the receptacle being positioned adjacent to the first open end; and
the second open end being positioned adjacent to the handle.

8. A tissue-sampling brush comprises:
an elongated body;
a receptacle;
a sheath;
the elongated body comprises a brush portion and a neck portion;
the neck portion comprises a first end and a second end;
the brush portion being connected adjacent to the first end;
the receptacle comprises a concave opening;
the receptacle being positioned adjacent to the brush portion opposite to the first end;
the concave opening being oriented towards the brush portion;
the sheath comprises a first open end and a second open end;
the first open end and the second open end being positioned opposite to each other along the sheath; and
the elongated body being sleeved by the sheath.

9. The tissue-sampling brush as claimed in claim 8 further comprises:
a handle; and
the handle being connected adjacent to the second end.

10. The tissue-sampling brush as claimed in claim 8 further comprises:
the brush portion comprises an elongated base and a plurality of bristles;
the plurality of bristles being radially positioned about the elongated base; and
the plurality of bristles being evenly distributed along the elongated base.

11. The tissue-sampling brush as claimed in claim 9 further comprises:
the handle comprises at least one finger loop, a loop hub, and a stem;
the stem being connected adjacent to the second end;
the loop hub being connected adjacent to the stem opposite to the second end; and
the at least one finger loop being connected about the loop hub.

12. The tissue-sampling brush as claimed in claim 9 further comprises:
wherein the brush portion, the neck portion, the handle, and the sheath are in a deployed configuration;
the brush portion being externally positioned to the sheath;
the brush portion being positioned adjacent to the first open end;
the first open end being positioned opposite of the receptacle along the brush portion; and
the second open end being positioned adjacent to the handle.

13. The tissue-sampling brush as claimed in claim 9 further comprises:
wherein the brush portion, the neck portion, the handle, and the sheath are in a retracted configuration;
the brush portion being internally positioned to the sheath;
the receptacle being positioned adjacent to the first open end; and
the second open end being positioned adjacent to the handle.

14. A tissue-sampling brush comprises:
an elongated body;
a receptacle;
a sheath;
a handle;
the elongated body comprises a brush portion and a neck portion;
the neck portion comprises a first end and a second end;
the brush portion being connected adjacent to the first end;
the receptacle comprises a concave opening;
the receptacle being positioned adjacent to the brush portion opposite to the first end;
the concave opening being oriented towards the brush portion;
the sheath comprises a first open end and a second open end;
the first open end and the second open end being positioned opposite to each other along the sheath;
the elongated body being sleeved by the sheath; and
the handle being connected adjacent to the second end.

15. The tissue-sampling brush as claimed in claim 14 further comprises:
the brush portion comprises an elongated base and a plurality of bristles;
the plurality of bristles being radially positioned about the elongated base; and
the plurality of bristles being evenly distributed along the elongated base.

16. The tissue-sampling brush as claimed in claim 14 further comprises:
the handle comprises at least one finger loop, a loop hub, and a stem;
the stem being connected adjacent to the second end;
the loop hub being connected adjacent to the stem opposite to the second end; and
the at least one finger loop being connected about the loop hub.

17. The tissue-sampling brush as claimed in claim 14 further comprises:
wherein the brush portion, the neck portion, the handle, and the sheath are in a deployed configuration;
the brush portion being externally positioned to the sheath;
the brush portion being positioned adjacent to the first open end;
the first open end being positioned opposite of the receptacle along the brush portion; and
the second open end being positioned adjacent to the handle.

18. The tissue-sampling brush as claimed in claim 14 further comprises:
wherein the brush portion, the neck portion, the handle, and the sheath are in a retracted configuration;
the brush portion being internally positioned to the sheath;
the receptacle being positioned adjacent to the first open end; and
the second open end being positioned adjacent to the handle.
